(19) United States
(12) Patent Application Publication
(10) Pub. No.: US 2003/0144352 A1
(43) Pub. Date: Jul. 31, 2003

(54) Antimuscarinic aerosol

(76) Inventors: Sue K. Cammarata, Portage, MI (US); Karen Kolbasa, Schoolcraft, MI (US); Joe Palandra, Kalamazoo, MI (US); Ivan Richards, Kalamazoo, MI (US); Mark P. Warchol, Kalamazoo, MI (US)

Correspondence Address: DINSMORE & SHOHL, LLP, 1900 CHEMED CENTER, 255 EAST FIFTH STREET, CINCINNATI, OH, 45202, US

(21) Appl. No.: 10/287,061

(22) Filed: Nov. 4, 2002

Related U.S. Application Data
(60) Provisional application No. 60/337,298, filed on Nov. 5, 2001.

(51) Int. Cl. A61K 31/215; A61K 31/137

(52) U.S. Cl. 514/531; 514/649

(57) ABSTRACT

The present invention concerns the use of antimuscarinic agents for the treatment of urinary disorders. The invention provides a method of treating urinary disorder in a mammal, including man, comprising administering to said mammal, in need of such a treatment, a therapeutically effective amount of an antimuscarinic agent, or solvate or prodrug thereof, said administration being performed by inhalation or insufflation.

Furthermore, the present invention provides a pharmaceutical composition for treating urinary disorder in a mammal, including man, which is in the form of an inhalable or insufflable preparation and comprises a therapeutically effective amount of an antimuscarinic agent, or solvate or prodrug thereof, together with an inhalably or insufflably acceptable carrier or diluent therefor.

The invention also provides a novel use of an antimuscarinic agent, or solvate or prodrug thereof, for the manufacture of an inhalable or insufflable medicament for therapeutical treatment of urinary disorders.

This application claims the benefit of U.S. Provisional Patent Application No. 60/337,298, filed Nov. 5, 2001, the entire disclosure of which is herein incorporated by reference.

### TECHNICAL FIELD

The present invention is within the field of urology. More specifically, it is generally based on the use of antimuscarinic agents for the treatment of urinary disorders, said antimuscarinic agents being administered by inhalation or insufflation.

### BACKGROUND OF THE INVENTION

Urinary disorders and symptoms thereof include some or all of the following: urgency, frequency, incontinence, urine leakage, enuresis, dysuria, hesitancy, and difficulty of emptying bladder. In particular, urinary disorders include urinary incontinence, caused by e.g. unstable or overactive urinary bladder.

A substantial part (5-10%) of the adult population suffers from urinary incontinence, and the prevalence, particularly of so-called urge incontinence, increases with age. The symptoms of an unstable or overactive bladder comprise urge incontinence, urgency and urinary frequency. It is assumed that unstable or overactive bladder is caused by uncontrolled contractions of the bundles of smooth muscle fibres forming the muscular coat of the urinary bladder (the detrusor muscle) during the filling phase of the bladder. These contractions are mainly controlled by cholinergic muscarinic receptors, and the pharmacological treatment of unstable or overactive bladder has been based on muscarinic receptor antiagonists.

The reason why the bladder muscle contracts inappropriately is unclear in many cases. For some people it may be due to a problem with the nerve signals that run from the brain to the bladder. Sometimes minor nerve damage is caused by surgery or childbearing. This muscle squeezes or contracts more often than normal and at inappropriate times. Instead of staying at rest as urine fills the bladder, the detrusor contracts while the bladder is filling with urine. This causes a person to feel a sudden and sometimes overwhelming urge to urinate even when the bladder is not full.

U.S. Pat. No. 5,382,600 discloses 2-[(1R)-3-diisopropylamino)-1-phenylpropyl)-4-methylphenol, also known as N,N-diisopropyl-3-(2-hydroxy-5-methylphenyl)-3-phenylpropylamine, with the generic name of tolterodine, as being useful to treat urinary incontinence. H Postlind et al, Drug Metabolism and Disposition, 26(4): 289-293 (1998) discloses that tolterodine is a muscarinic receptor antagonist. It is presently being sold in a number of different countries for treatment of urinary incontinence under the name Detrol®, marketed by Pharmacia. When tolterodine is used to treat urinary incontinence it is administered perorally as a tablet, The major, active metabolite of tolterodine is the 5-hydroxymethyl derivative of tolterodine.

U.S. Pat. No. 5,559,269 and H Postlind et al, Drug Metabolism and Disposition, 26(4): 289-293 (1998) disclose hydroxytolterodine. U.S. Pat. No. 5,559,269 discloses this compound as being useful to treat urinary incontinence. Pharmacol. Toxicol., 81: 169-172 (1997) discloses that hydroxytolterodine has antimuscarinic activity. The international patent application WO 02/34245 discloses the use of tolterodine for treating asthma, COPD, and allergic rhinitis.

The international patent application WO 98/43942 discloses therapeutically active diarylpropylamines, which have favorable anticholinergic properties, and which can be used for the treatment of disorders related to urinary incontinence.

U.S. Pat. No. 6,124,354 discloses 2-diisopropylamino)ethyl-1-phenylcyclopentanecarboxylate and its use in treating urinary incontinence and irritable bowel syndrome (see Example 99). Can. J. Chem., 40: 1909-1916 (1962) refers to this compound as a potential antidote for treatment of anticholinesterase poisoning, J. Am. Chem. Soc., 69. 2902-2906 (1947), while not mentioning the diisopropylamino compound but a diethylamino analog, discloses that the diethylamino compound has antispasmolytic action against acetylcholine.

While efficiently relieving urinary incontinence in affected patients, the above-mentioned commercially available compounds do not provide their effects instantly upon administration thereof to the patient. Since urinary disorder symptoms often have a rapid onset, it is desirable to relieve the symptoms instantly.

The currently marketed administration form of tolterodine is film-coated tablets containing 1 mg, 2 mg or 4 mg of tolterodine L-tartrate for release in the gastrointestinal tract. Consumers constantly require alternative delivery forms, especially when the need for medicament treatment is urgent and/or when the patient has an active life-style.

Hence, known treatments are insufficient to certain groups of patients, which demand a more flexible treatment to meet their active way of life.

There is a need for new delivery forms of antimuscarinic agents for treatment of urinary disorders, which delivery forms possess properties such that the mentioned problems can be overcome.

### SUMMARY OF THE INVENTION

For these and other purposes, it is an object of the present invention to provide a method of treating urinary disorder in a mammal, including man, which method brings instant relief from symptoms arising from said urinary disorder.

It is also an object of the present invention to provide a method of treating urinary disorder in a mammal, including man, which method involves alternative delivery forms that are particularly suitable for urgent or acute treatment of symptoms.

It is an object of the present invention to provide a method of treating urinary disorder, in a mammal, including man, which method is compatible with an active life-style.

It is a further object of the present invention to provide a pharmaceutical composition for treating urinary disorder in a mammal, including man, which can bring instant relief from symptoms arising from said urinary disorder.

It is also an object of the present invention to provide a pharmaceutical composition for treating urinary disorder in a mammal, including man, which is appropriate for alternative delivery forms being particularly suitable for urgent or acute treatment of symptoms.

It is an object of the present invention to provide a pharmaceutical composition for treating urinary disorder in a mammal, including man, use of which is compatible with an active life-style.

Another object of the present invention is to provide a novel use of an agent active against urinary disorder for the manufacture of a medicament for therapeutical treatment of urinary disorders, which medicament can bring instant relief from symptoms arising from said urinary disorder.

It is also an object of the present invention to provide a novel use of an agent active against urinary disorder for the manufacture of a medicament for therapeutical treatment of urinary disorders, which medicament is appropriate for alternative delivery forms that are particularly suitable for urgent or acute treatment of symptoms.

Yet another object of the present invention is to provide a novel use of an agent active against urinary disorder for the manufacture of a medicament for therapeutical treatment of urinary disorders, which medicament is compatible with an active life-style.

For these and other objects which will be evident from the following disclosure, the present invention provides a method of treating urinary disorder in a mammal, including man, comprising administering to said mammal, in need of such a treatment, a therapeutically effective amount of an antimuscarinic agent, or solvate or prodrug thereof, said administration being performed by inhalation or insufflation.

The invention is based on the insight that antimuscarinic agents are rapidly distributed to the systemic circulation upon delivery via inhalation or insufflation, thus providing their effects instantly at target organs, such as the smooth muscles regulating emptying of the urinary bladder.

In one preferred embodiment of the method according to the invention, said disorder is unstable or overactive urinary bladder.

In a preferred embodiment of the method according to the invention, said disorder is urinary incontinence.

In another preferred embodiment of the method according to the invention, said antimuscarinic agent, or solvate or prodrug thereof, is administered as an aerosol formulation.

In yet another, preferred embodiment of the method according to the invention, said antimuscarinic agent, or solvate or prodrug thereof, is administered as a powder formulation.

In a preferred embodiment of the method according to the invention, said antimuscarinic agent, or solvate or prodrug thereof, is selected from the group consisting of 3,3-diphenylpropylamines and arylcycloalkane carboxylic esters, and inhalably or insufflably acceptable salts thereof.

In a more preferred embodiment of the method according to the invention, said antimuscarinic agent is selected from the group consisting of tolterodine, hydroxytolterodine, and 2-(diisopropylamino)ethyl-1-phenylcyclopentanecarboxylate, as well as inhalably or insufflably acceptable salts thereof.

In a more preferred embodiment of the method according to the invention, said antimuscarinic agent is selected from the group consisting of tolterodine and inhalably or insufflably acceptable salts thereof.

In the most preferred embodiment of the method according to the invention, said antimuscarinic agent is selected from the group consisting of tolterodine and tolterodine L-tartrate.

In a preferred embodiment of the method according no the invention, the administered amount of said antimuscarinic agent is from about 0.05 mg to about 12 mg.

In a more preferred embodiment of the method according to the invention, the administered amount of said antimuscarinic agent is from about 0.1 to about 6 mg.

In the most preferred embodiment of the method according to the invention, the administered amount of said antimuscarinic agent is from about 0.2 to about 5 mg.

Furthermore, the present invention provides a pharmaceutical composition for treating urinary disorder in a mammal, including man, which is in the form of an inhalable, or insufflable preparation and comprises a therapeutically effective amount of an antimuscarinic agent, or solvate or prodrug thereof, together with an inhalably or insufflably acceptable carrier or diluent therefor.

In one preferred embodiment of the composition according to the invention, said disorder is unstable or overactive urinary bladder.

In a preferred embodiment of the composition according to the invention, said disorder is urinary incontinence.

In another preferred embodiment of the composition according to the invention, said composition is an aerosol formulation.

In yet another preferred embodiment of the composition according to the invention, said composition is a powder formulation.

In one preferred embodiment of the composition according to the insertion, said antimuscarinic agent, or solvate or prodrug thereof, is selected from the group consisting of 3,3-diphenylpropylamines and arylcycloalkane carboxylic esters, and inhalably or insufflably acceptable salts thereof.

In a more preferred embodiment of the composition according to the invention, said antimuscarinic agent is selected from the group consisting of tolterodine, hydroxytolterodine, and 2-(diisopropylamino)ethyl-1-phenylcyclopentanecarboxylate, as well as inhalably or insufflably acceptable salts thereof.

In a more preferred embodiment of the composition according to the invention, said antimuscarinic agent is selected from the group consisting of tolterodine and inhalably or insufflably acceptable salts thereof.

In the most preferred embodiment of the composition according to the invention, said antimuscarinic agent is selected from the group consisting of tolterodine and tolterodine L-tartrate.

In a preferred embodiment of the composition according to the invention, said antimuscarinic agent is present in an amount of from about 0.05 mg to about 12 mg, preferably from about 0.1 to about 6 mg, and more preferably from about 0.2 to about 5 mg.

The present invention also provides a novel use of an antimuscarinic agent, or solvate or prodrug thereof, for the manufacture of an inhalable or insufflable medicament for therapeutical treatment of urinary disorders.

In one preferred embodiment of the use according to the invention, said disorder is unstable or overactive urinary bladder.

In a preferred embodiment of the use according to the invention, said disorder is urinary incontinence.

In another preferred embodiment of the use according to the invention, said medicament is an aerosol formulation.

In yet another preferred embodiment of the use according to the invention, said medicament is a powder formulation.

In a preferred embodiment of the use according to the invention, said antimuscarinic agent, or solvate or prodrug thereof, is selected from the group consisting of 3,3-diphenylpropylaminies and arylcycloalkane carboxylic esters, and inhalably or insufflably acceptable salts thereof.

In a more preferred embodiment of the use according to the invention, said antimuscarinic agent is selected from the group consisting of tolterodine, hydroxytolterodine, and 2-(diisopropylamino)ethyl-1-phenylcyclopentanecarboxylate, as well as inhalably or insufflably acceptable salts thereof.

In a more preferred embodiment of the use according to the invention, said antimuscarinic agent is selected from the group consisting of tolterodine and inhalably or insufflably acceptable salts thereof.

In the most preferred embodiment of the use according to the invention, said antimuscarinic agent is selected from the group consisting of tolterodine and tolterodine L-tartrate.

### BRIEF DESCRIPTION OF THE DRAWINGS

FIG. 1 is a diagram showing the plasma concentration (ng/ml)of tolterodine with time (hours) upon systemic and local administration (aerosol) in mice.

FIG. 2 is a diagram showing the plasma concentration (ng/ml) of tolterodine with time (hours) upon local administration (aerosol) of various amounts in mice.

FIG. 3 is a diagram showing the variation of serum concentration (nmol/l) of tolterodine and its active metabolite with time (hours) during 9 hours upon administration of tolterodine perorally through a 2 mg tablet in human patients.

### DESCRIPTION OF THE INVENTION

The present invention involves the use of antimuscarinic agents to treat urinary disorders, such as unstable or overactive urinary bladder.

Overactive urinary bladder encompasses various urinary disorders, including overactive urinary bladder detrusor instability, detrusor hyperreflexia, urge incontinence, urgency and urinary frequency and LUTS (Lower Urinary Tract Symptoms giving obstructive urinary symptoms such as slow urination, dribbling at the end of urination, inability to urinate and/or the need to strain to urinate at an acceptable rate or irritate symptoms such as frequency an/or urgency).

Other conditions are also included, which give rise to urinary frequency, urgency and/or urge incontinence. Overactive bladder disorders also include nocturia and mixed incontinence. While overactive bladder is often associated with detrusor muscle instability, disorders of bladder function may also be due to neuropathy of the central nervous system (detrusor hyperreflexia) including spinal cord and brain lesions, such as multiple sclerosis and stroke. Overactive bladder symptoms may also result from, for example, male bladder outlet obstruction (usually due to prostatic hypertrophy), interstitial cystitis, local edema and irritation due to focal bladder cancer, radiation cystitis due to radiotherapy to the pelvis, and cystitis.

The method of the present invention is used to treat mammals, including man. It is preferred that the mammal is a human.

Upon traditional tablet administration of antimuscarinic agents to treat urinary disorders, the plasma concentration thereof increases rather slowly, peaking after 1-2 hours. The antimuscarinic agents are often metabolized by the liver following oral dosing. According to the present invention, administration of antimuscarinic agents to patients for treatment of urinary disorders can advantageously be performed via inhalation or insufflation. Thereby, the antimuscarinic agents instantly gain access to the systemic circulation and can affect target tissues, such as the smooth musculature surrounding the urinary tract.

The compositions according to the invention can be made up in solid or liquid form, such as powders, sterile solutions, suspensions or emulsions, and the like.

The antimuscarinic agents of the present invention are administered by inhalation or insufflation. The inhalation or insufflation is preferably by either an aerosol or a powder.

The method and the antimuscarinic agents and compositions of the present invention are useful for the treatment of unstable or overactive urinary bladder, e.g. urinary incontinence.

The dosage of the specific antimuscarinic agent will vary depending on its potency, the mode of administration, the age and weight of the patient and the severity of the condition to be treated. The daily dosage may, for example, range from about 0.01 mg to about 4 mg per kg of body weight, administered singly or multiply in doses e.g. from about 0.05 mg to about 200 mg each. A clinically effective amount of antimuscarinic agents is from about 0.05 mg to about 1.2 mg. It is preferred that the effective amount is from about 0.1 to about 6 mg; it is more preferred that the effective amount is from about 0.2 to about 5 mg.

The dosage form for inhalation can be an aerosol. The minimum amount of an aerosol delivery is about 0.2 ml and the maximum aerosol delivery is about 5 ml. The concentration of the antimuscarinic agents may vary as long as the total amount of spray delivered is within the about 0.2 to about 5 ml amount and it delivers an effective amount. It is well known to those skilled in the art that if the concentration is higher, one gives a smaller dose to deliver the same effective amount.

The non-active ingredient or carrier can be just (sterile) water with the pH adjusted to where the active pharmaceutical agent is very soluble. It is preferred that the pH be at or near 7. Alternatively and preferably, the non-active carrier agent should be physiological saline with the pH adjusted appropriately. Aerosols for inhalation of various pharmaceutical agents are well known to those skilled in the art, including many aerosols for treating asthma.

Alternatively, the dosage form for inhalation can be powder. Powders for inhalation of various pharmaceutical agents are well known to those skilled in the art, including many powders for treating asthma. When the dosage form is a powder, the antimuscarinic agent can be administered in pure form or diluted with an inert carrier. When an inert carrier is used, the antimuscarinic agent is compounded such that the total amount of powder delivered delivers an “effective amount” of the agent. The actual concentration of the agent may vary. If the concentration is lower, then more powder must be delivered; if the concentration is higher, less total material must be delivered to provide an effective amount of the agent.

The carriers may be of any inert material, organic or inorganic, suitable for administration via inhalation or insufflation, such as: water, gelatin, gum arabicum, lactose, microcrystalline cellulose, starch, sodium starch glycolate, calcium hydrogen phosphate, magnesium stearate, talcum, colloidal silicon dioxide, and the like. Such compositions may also contain other pharmaceutically active agents, and conventional additives, such as stabilizers, wetting agents, emulsifiers, flavoring agents, buffers, and the like.

Various devices are on the market for administering powders for inhalation for asthma, and these devices are suitable for administering the antimuscarinic agents of the present invention.

Pharmaceutically acceptable salts include salts of both inorganic and organic acids. The pharmaceutically acceptable salts are preferred over the corresponding free amities since they produce compounds that are more water soluble and more crystalline. The preferred pharmaceutically acceptable salts include salts of the following acids: tartaric, hydrochloric, hydrobromic, sulfuric, phosphoric nitric, citric, methanesulfonic, CH₃—(CH₂)ₙ—COOH where n is 0 through 4, HOOC—(CH₂)ₙ—COOH, where n is as defined above, HOOC—CH═CH—COOH, φ—COOH. For other acceptable salts, see Int. J. Pharm., 33: 201-217 (1986).

An exemplary class of antimuscarinic agents which may be used as active ingredients in the present invention comprises the arylcycloalkane carboxylic esters disclosed in U.S. Pat. No. 6,124,354 (the entire disclosures of which are incorporated by reference herein).

An exemplary specific antimuscarinic agent is 2-[bis(1-methylethyl)amino]ethyl-1-phenylcyclopentanecarboxylate, also known as 2-(diisopropylamino)ethyl-1-phenylcyclopentanecarboxylate, as well as metabolizes, prodrug forms and pharmaceutically acceptable salts thereof.

Another exemplary class of antimuscarinic agents which may be used as active ingredients in the present invention comprises the 3,3-diphenylpropylamines disclosed in U.S. Pat. Nos. 5,382,600, 5,559,269 and 5,686,464 (the entire disclosures of which are incorporated by reference herein) and having the general formula:

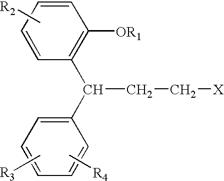

wherein R₁ signifies hydrogen or methyl; R₂, R₃ and R₄ independently signify hydrogen, methyl, methoxy, hydroxy, hydroxymethyl, carbamoyl, sulphamoyl or halogen; and X represents a tertiary amino group —NR₅R₆, wherein R₅ and R₆ signify non-aromatic hydrocarbyl groups, which may be the same or different, especially C₁₋₆-alkyl or adamantyl, and which together contain at least three, preferably at least four carbon atoms, and each of which may carry a hydroxy substituent, and wherein R₅ and R₆ may form a ring together with the amine nitrogen, preferably a non-aromatic ring having no heteroatom other than the amine nitrogen, their salts with physiologically acceptable acids and, when the compounds can be in the form of optical isomers, the racemic mixture and the individual enantiomers.

Exemplary specific compounds include tolterodine, i.e. (R)-N,N-diisopropyl-3-(2-hydroxy-5-methylphenyl)-3-phenylpropanamine, as well as the corresponding (S)-enantiomer, the racemate and the active 5-hydroxymethyl metabolites, solvates, prodrug forms and pharmaceutically acceptable salts thereof.

Useful analogues to the above compounds are disclosed in WO 98/43942 (the full disclosure of which is incorporated by reference herein).

Specifically, the compositions according to the present invention have proved to be very suitable for administering the above-mentioned drug tolterodine and would likewise be suitable for its related compounds, i.e. the major, active metabolite of tolterodine, i.e. (R)-N,N-diisopropyl-3-(2-hydroxy-5-hydroxymethylphenyl)-3-phenylpropanamine; the corresponding (S)-enantiomer to tolterodine, i.e. (S)-N,N-diisopropyl-3-(2-hydroxy-5-methylphenyl)-3-phenylpropanamine; the 5-hydroxymethyl metabolite of the (S)-enantiomer, i.e. (S)-N,N-diisopropyl-3-(2-hydroxy-5-hydroxymethylphenyl)-3-phenylpropanamine; as well as the corresponding racemate to tolterodine, i.e. (R,S)-N,N-diisopropyl-3-(2-hydroxy-5-methylphenyl)-3-phenylpropanamine; and prodrug forms and pharmacologically acceptable salts thereof.

Tolterodine refers to 2-[(1R)-3-(diisopropylamino)-1-phenylpropyl]-4-methylphenol, also known as (R)-N,N-diisopropyl-3-(2-hydroxy-5-methylphenyl)-3-phenylpropylamine, a compound of the formula:

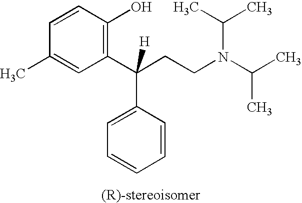

Hydroxytolterodine refers to 2-[(1R)-3-(diisopropylamino)-1-phenylpropyl]-4-(hydroxymethyl)phenol, a compound of the formula:

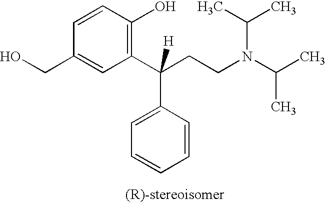

2- [bis(1-methylethyl)amino]ethyl-1-phenylcyclopentanecarboxylate, also known as 2-(diisopropylamino)ethyl-1-phenylcyclopentanecarboxylate, refers to a compound of the formula:

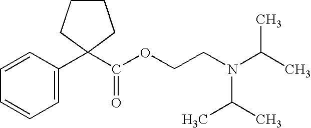

“Antimuscarinic agents” refer to muscarinic receptor antagonists. Examples of antimuscarinic agents include, but are not limited to, tolterodine, hydroxytolterodine, 2-(diisopropylamino)ethyl-1-phenylcyclopentanecarboxylate, propiverine, oxybutynin, trospium, darifenacin, temiverine, and ipratropium.

Propiverine is 1-methyl-4-piperidyl .alpha., .alpha.- diphenyl-.alpha-(n-propoxy)acetate and is disclosed in East German Patent 106,643 and in CAS 82-155841s (1975). Oxybutynin is 4-(diethylamino)-2-butynylalphaphenylcyclohexaneglycolate and is disclosed in UK Patent 940,540. Trospium is 3alpha-hydroxyspiro [1alphaH,5alphaH-nortropane-8,1′pyrrolidinium]chloride benzilate and is disclosed in U.S. Pat. No. 3,480,623. Darifenacin is 3-Pyrrolidineacetamide, 1-[2-(2,3-dihydro-5-benzofuranyl)ethyl]-alpha,alpha-diphenyl-, and is disclosed in U.S. Pat. No. 5,096,890. Temivorine is benzeneacetic acid, alpha.-cyclohexyl-.alpha.-hydroxy-, 4-(diethylamino)-1,1-dimethyl-2-butynyl ester and is disclosed in U.S. Pat. No. 5,035,098. Ipratropium is 8-isopropylnoratropine methobromide and is disclosed in U.S. Pat. No. 3,505,337.

“Physiological saline” generally refers to a 0.9% aqueous sodium chloride solution.

“Pharmaceutically acceptable” refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability.

Analogously, “inhalably acceptable” and “insufflably acceptable”, respectively, refer to properties and/or substance which are pharmaceutically acceptable and also suitable for use via inhalation and insufflation, respectively.

### EXAMPLES

Without further elaboration, it is believed that one skilled in the art can, using the preceding description, practice the present invention to its fullest extent. The following detailed examples describe how to prepare the various antimuscarinic agent and/or perform the various methods of the invention and are to be construed as merely illustrative, and not limitations of the preceding disclosure in any way whatsoever. Those skilled in the art will promptly recognize appropriate variations from the procedures both as to reactants and as to reaction conditions and techniques.

### Example 1

### Pharmacokinetic Comparison of Systemic and Local (Aerosol) Administration, Respectively, of Tolterodine

Female BALB/C mice, weight range 19-22 g, were obtained from Charles River Laboratories (Kingston, N.C.). They received food and water ad libitum. All procedures in these studies were in compliance with the Animal Welfare Act Regulation, 9CFR Parts 1 and 2, Publication (NIH) 85-23, 1985.

Tolterodine L-tartrate, i.e. (R)-N,N-diisopropyl-3-(2-hydroxy-5-methylphenyl)-3-phenylpropanamine L-tartrate, for intraperitoneal administration was prepared in sterile 0.9% NaCl.

Tolterodine L-tartrate for aerosol administration was prepared in sterile phosphate buffer solution at a concentration of 1.0 mg/ml.

Mice were placed in a carousel-style, nose only, exposure chamber and allowed to inhale aerosols of tolterodine for five minutes, using an ICN SPAG-2 nebulizer. This nebulizer generates a mean aerosol particle size of 1.3 microns at a rate of approximately 0.25 ml/minute.

Thus, mice received tolterodine either by aerosol generated from a 1 mg/ml solution for five minutes or by intraperitoneal (i.p.) injection at a dose of 3 mg/kg. Blood samples were taken via cardiac puncture under isoflurane anesthesia at 5, 15, 30, 60, 120, and 240 minutes after in treatment and at 2.5, 5, 15, 30, 60, and 120 minutes after aerosol drug treatment.

The samples were collected in tubes containing EDTA and centrifuged at 12000×g for four minutes. Plasma was removed and stored at −70° C. until assay.

Plasma samples were extracted via a liquid/liquid extraction technique. Plasma levels for tolterodine were determined by ESI-LC/MS/MS using a PE SCIEX API 3000 mass spectrometer in positive ion mode. Chromatographically, the analyte and internal standard were resolved on a Zorbax ACE Phenyl column (2.1×50mm) using a gradient elution. The total analysis time was 4 minutes with a limit of quantization of 100 pg/mL.

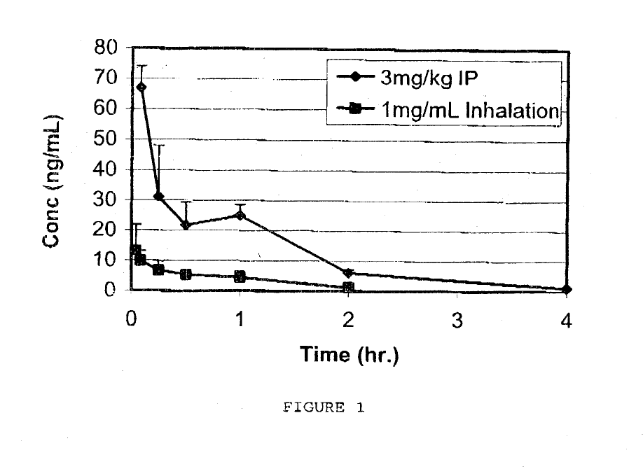

Plasma concentrations of tolterodine following 3 mg/kg i.p. injection and following 1 mg/ml aerosol exposure (inhalation) are summarized in FIG. 1.

### Example 2

### Aerosol Administration of Different Amounts of Tolterodine

Female BALB/c mice, weight range 19-22 g, were obtained from Charles River Laboratories (Kingston, N.C.). They received food and water ad libitum. All procedures in these studies were in compliance with the Animal Welfare Act Regulation, 9CFR Parts 1 and 2, Publication (NIH) 85-23, 1985.

Tolterodine L-tartrate for aerosol administration was prepared in sterile phosphate buffer solution at concentrations of 0,1, 0.5, and 1.0 mg/ml.

As described in Example 1, mice were exposed to aerosols of tolterodine generated from either 0.1, 0.5, or 1.0 mg/ml solutions. The duration of aerosol treatment was five minutes. Blood samples were collected via cardiac puncture at 2.5, 5, 15, 30, 60, and 120 minutes following the end of the drug nebulization period.

The samples from were collected in tubes containing EDTA and centrifuged at 12000×g for four minutes. Plasma was removed and stored at −70° C. until assay.

Plasma samples were extracted and plasma levels for tolterodine were determined as described in Example 1.

FIG. 2 shows plasma concentrations of tolterodine L-tartrate following inhalation of nebulized solutions at 0.1, 0.5, or 1.0 mg/mL. Plasma levels for the 0.1 mg/mL concentration were at or below-detection limits. Clearly, tolterodine is rapidly absorbed into the circulation.

### Example 3

### Comparative Pharmacokinetic Study of Oral Administration of Tolterodine

This example illustrated the systemic distribution in man of perorally administrated prior art tolterodine tablets.

In 30 human patients with overactive bladder, the pharmacokinetic effects were determined of a film-coated tablet containing 2 mg of tolterodine L-tartrate. Serum concentrations of tolterodine and its main 5-hydroxymethyl metabolite (below called 5-HM) were measured over time.

Blood samples were drawn immediately before dosing and after 0.5, 1, 2, 3, 6 and 9 hours, and the free (unbound) serum concentrations of tolterodine and its 5-HM metabolite were measured by gas chromatography/mass spectrometry. The unbound concentrations were calculated assuming a fraction unbound of 3.7% for tolterodine and of 36% for 5-HM as obtained from protein binding studies on human serum (Nilvebrant, L., et al., Life Sciences, Vol. 60, Nos. 13/14 (1997) 1129-1136).

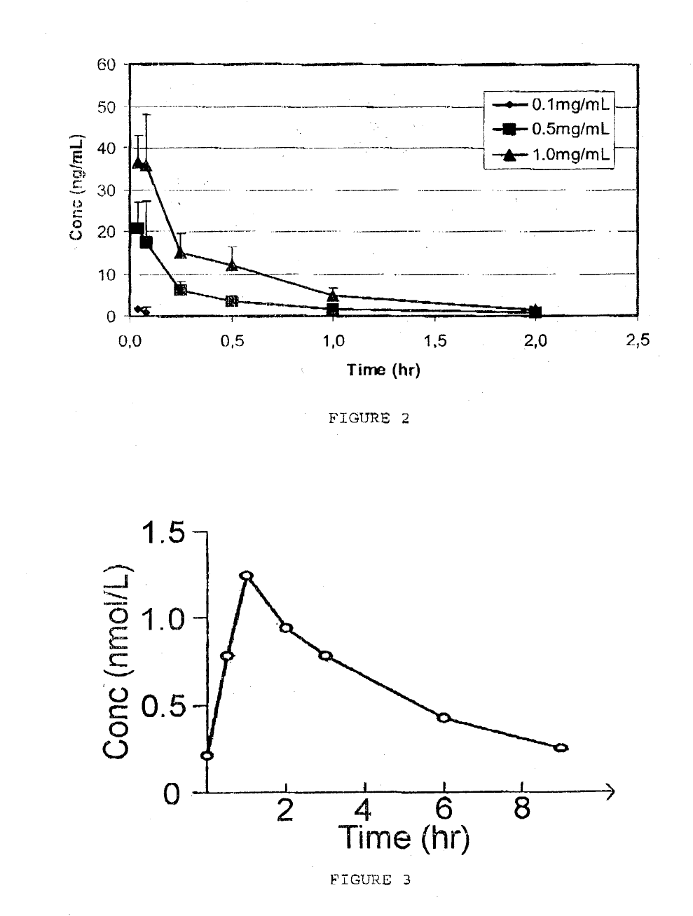

FIG. 3 shows the obtained variation with time of the sum of the unbound concentrations of tolterodine and 5-HM for the administration of a 2 mg tablet.

It is apparent that the patterns of blood concentrations of tolterodine and its active metabolite are altered upon aerosol administration thereof (examples 1 and 2, FIGS. 1 and 2), when compared to prior art oral administration (example 3, FIG. 3). Aerosol administration (FIGS. 1 and 2) produces within a few minutes a distinct and instant rise in tolterodine plasma concentration, similar in pattern to what is seen upon intraperitoneal injection (FIG. 1). In contrast, oral administration (FIG. 3) results in slower uptake of tolterodine into the circulation, wherein a maximum blood concentration is reached in the range of one hour, and a concomitant prolonged presence of tolterodine in the circulation.

1. A method of treating urinary disorder in a mammal, including man, comprising administering to said mammal, in need of such a treatment, a therapeutically effective amount of an antimuscarinic agent, or solvate or prodrug thereof, said administration being performed by inhalation or insufflation.

2. A method according to claim 1, wherein said disorder is unstable or overactive urinary bladder.

3. A method according to claim 1, wherein said disorder is urinary incontinence.

4. A method according to claim 1, wherein said antimuscarinic agent, or solvate or prodrug thereof, is administered as an aerosol formulation.

5. A method according to claim 1, wherein said antimuscarinic agent, or solvate or prodrug thereof, is administered as a powder formulation.

6. A method according to any one of claims 1-5, wherein said antimuscarinic agent, or solvate or prodrug thereof, is selected from the group consisting of 3,3-diphenylpropylamines and arylcycloalkane carboxylic esters, and inhalably or insufflably acceptable salts thereof.

7. A method according to claim 6, wherein said antimuscarinic agent is selected from the group consisting of tolterodine, hydroxytolterodine, and 2-(diisopropylamino)ethyl-1-phenylcyclopentanecarboxylate, as well as inhalably or insufflably acceptable salts thereof.

8. A method according to claim 7, wherein said antimuscarinic agent is selected from the group consisting of tolterodine and inhalably or insufflably acceptable salts thereof.

9. A method according to claim 8, wherein said antimuscarinic agent is selected from the group consisting of tolterodine and tolterodine L-tartrate.

10. A method according to claim 1, wherein the administered amount of said antimuscarinic agent is from about 0.05 mg to about 12 mg.

11. A method according to claim 1, wherein the administered amount of said antimuscarinic agent is from about 0.1 to about 6 mg.

12. A method according to claim 1, wherein the administered amount of said antimuscarinic agent is from about 0.2 to about 5 mg.

13. A pharmaceutical composition for treating urinary disorder in a mammal, including man, which is in the form of an inhalable or insufflable preparation and comprises a therapeutically effective amount of an antimuscarinic agent, or solvate or prodrug thereof, together with an inhalably or insufflably acceptable carrier or diluent therefor.

14. A composition according to claim 13, wherein said disorder is unstable or overactive urinary bladder.

15. A composition according to claim 13, wherein said disorder is urinary incontinence.

16. A composition according to claim 13, which is an aerosol formulation.

17. A composition according to claim 13, which is a powder formulation.

18. A composition according to any one of claims 13-17, wherein said antimuscarinic agent, or solvate or prodrug thereof, is selected from the group consisting of 3,3-diphenylpropylamines and arylcycloalkane carboxylic esters, and inhalably or insufflably acceptable salts thereof.

19. A composition according to claim 18, wherein said antimuscarinic agent is selected from the group consisting of tolterodine, hydroxytolterodine, and 2-(diisopropylamino)ethyl-1-phenylcyclopentanecarboxylate, as well as inhalably or insufflably acceptable salts thereof.

20. A composition according to claim 19, wherein said antimuscarinic agent is selected from the group consisting of tolterodine and inhalably or insufflably acceptable salts thereof.

21. A composition according to claim 20, wherein said antimuscarinic agent is selected from the group consisting of tolterodine and tolterodine L-tartrate.

22. A composition according to claim 13, wherein said antimuscarinic agent is present in an amount of from about 0.05 mg to about 12 mg.

23. A composition according to claim 13, wherein said antimuscarinic agent is present in an amount of from about 0.1 to about 6 mg.

24. A composition according to claim 13, wherein said antimuscarinic agent is present in an amount of from about 0.2 to about 5 mg.

25. Use of an antimuscarinic agent, or solvate or prodrug thereof, for the manufacture of an inhalable or insufflable medicament for therapeutical treatment of urinary disorders.

26. Use according to claim 25, wherein said disorder is unstable or overactive urinary bladder.

27. Use according to claim 25, wherein said disorder is urinary incontinence.

28. Use according to claim 25, wherein said medicament is an aerosol formulation.

29. Use according to claim 25, wherein said medicament is a powder formulation.

30. Use according to any one of claims 25-29, wherein said antimuscarinic agent, or solvate or prodrug thereof is selected from the group consisting of 3,3-diphenylpropylamines and arylcycloalkane carboxylic esters, and inhalably or insufflably acceptable salts thereof.

31. Use according to claim 30, wherein said antimuscarinic agent is selected from the group consisting of tolterodine, hydroxytolterodine, and 2-(diisopropylamino)ethyl-1-phenylcyclopentanecarboxylate, as well as inhalably or insufflably acceptable salts thereof.

32. Use according to claim 31, wherein said antimuscarinic agent is selected from the group consisting of tolterodine and inhalably or insufflably acceptable salts thereof.

33. Use according to claim 32, wherein said antimuscarinic agent is selected from the group consisting of tolterodine and tolterodine L-tartrate.
